(19) United States
(12) Patent Application Publication
(10) Pub. No.: US 2002/0068859 A1
(43) Pub. Date: Jun. 6, 2002

(54) Laser diode drive scheme for noise reduction in photoplethysmographic measurements

(76) Inventor: Christina A. Knopp, Amesbury, MA (US)

Correspondence Address: MARSH FISCHMANN & BREYFOGLE LLP, 3151 South Vaughn Way, Suite 411, Aurora, CO, 80014, US

(21) Appl. No.: 09/727,562

(22) Filed: Dec. 1, 2000

(51) Int. Cl. A61B 5/00

(52) U.S. Cl. 600/322

(57) ABSTRACT

The present invention discloses a photoplethysmographic measurement apparatus and related method for determining a blood analyte level in a tissue under test employing an inventive laser diode drive scheme to achieve noise reduction. Noise reduction is achieved by driving a plurality of laser diodes with modulated drive signals to cause emission of light signals from the laser diodes that are directed through the tissue under test and from which various blood analyte levels are determinable based upon the intensities of the transmitted light signals. Each drive signal is modulated at an appropriate modulation frequency that causes its corresponding laser diode to operate in a low noise regime wherein laser intensity noise is reduced, and the modulation depth of each drive signal is set to broaden the line width of the laser diode and thereby reduce the potential for optical feedback noise. In this regard, the modulation frequency and depth of each drive signal may be set to achieve operation of its corresponding laser diode at a desired laser intensity noise level. The desired laser intensity noise level may be near that (e.g., within the same order of magnitude) of the independent laser RIN level.

### FIELD OF THE INVENTION

The present invention generally relates to the field of photoplethysmography, and more particularly, to noise reduction in photoplethysmographic systems.

### BACKGROUND OF THE INVENTION

Photoplethysmography involves the transmission of light signals through a tissue under test to non-invasively determine the level of one or more blood analytes. More specifically, photoplethysmographic devices are used to determine concentrations of blood analytes such as oxyhemoglobin (O2Hb), deoxyhemoglobin or reduced hemoglobin (RHb), carboxyhemoglobin (COHb) and methemoglobin (MetHb) in a patient's blood.

One type of photoplethysmographic device includes a probe having a plurality of light signal sources (e.g., four light-emitting-diodes (LED's) or laser diodes) and one detector (e.g., a light sensitive photodiode). The probe is releasably attached to a patient's appendage (e.g., finger, ear lobe, nasal septum, or foot). Light signals characterized by distinct center wavelengths λ₁≠λ₂≠λ₃≠λ₄ emitted from the sources are directed through the appendage to the detector. The detector detects the transmitted light signals (light exiting the appendage is referred to as transmitted) and outputs a signal indicative of the intensity of the transmitted light signals. Since the different analytes have unique light absorbency characteristics, the signal output from the detector may be used to determine the concentrations of the blood analytes. See, e.g., U.S. Pat. No. 5,842,979, hereby incorporated by reference in its entirety.

When only one detector is used to detect the transmitted light signals, the signal output by the detector is comprised of signals corresponding to the four different transmitted light signals. Thus, a multiplexing method is typically employed so that the intensities of the four different transmitted light signals may be obtained (i.e. demultiplexed) from the multiplexed output signal. For example, a time-division multiplexing method may be employed wherein the different sources are pulsed (i.e. turned on then off) at different predetermined or monitored times during a repeated cycle so that the multiplexed output signal can be demultiplexed based on the known or monitored transmission times of each source. See, e.g., U.S. Pat. No. 5,954,644, hereby incorporated by reference in its entirety. Another example is a frequency-division multiplexing method wherein each of the different sources are pulsed at different frequencies so that the multiplexed output signal can be demultiplexed based on the frequency of pulses corresponding with each source. See, e.g., U.S. Pat. No. 4,800,885, hereby incorporated by reference in its entirety.

As may be appreciated, noise in the multiplexed output signal can reduce accuracy when determining the different blood analyte concentrations. One source of noise may be the light signal sources. While the incoherent output of an LED makes it relatively insensitive to optical feedback, this is not the case with a laser diode. The highly coherent output of a laser diode makes it susceptible to optical feedback, which can in turn increase the noise floor of the operating laser diode thereby introducing instabilities resulting from optical feedback (i.e., optical feedback noise) into the light signal emitted therefrom.

Another source of noise results from heating of the laser diode junction during the time that the laser diode is on (i.e., as the drive signal is applied). The length of a laser diode cavity increases with increasing laser diode temperature, thereby causing changes in the resonant conditions of the laser diode such that the wavelength of the light signal emitted from the laser diode changes over the time that the laser is on. In a semiconductor laser diode, the wavelength versus temperature characteristic curve has discontinuities which translate into hops in laser wavelength on the order of several angstroms (i.e., mode hopping) with increasing temperature. Mode hopping can introduce noise in two ways. First, a change in the wavelength of a transmitted light signal during measurement of a blood analyte level may be indistinguishable from a change in the level of the blood analyte being measured. Second, mode hopping also causes a discontinuity in laser output power thereby introducing laser intensity noise in the emitted light signal.

### SUMMARY OF THE INVENTION

Accordingly, the present invention provides a photoplethysmographic measurement apparatus and method that achieves increased accuracy in various blood analyte determinations by reducing laser noise in the light signals used to determine the various blood analyte levels. Noise reduction is accomplished by driving each laser diode light signal emitter of the photoplethysmographic apparatus with a corresponding modulated drive signal having an appropriate modulation frequency and modulation depth.

According to one aspect of the present invention a photoplethysmographic measurement apparatus for determining a blood analyte level in a tissue under test includes a plurality of laser diodes, a detector, a drive signal generator and a demodulator. The laser diodes are operable to transmit a corresponding plurality of light signals centered at different predetermined wavelengths through the tissue under test in response to a corresponding plurality of drive signals. In one embodiment, the apparatus of the present invention includes first and second laser diodes. The first laser diode is operable to transmit a first light signal centered at a first predetermined wavelength in the range of 600 nm to 700 nm and the second laser diode is operable to transmit a second light signal centered at a second predetermined wavelength in the range of 900 nm to 1000 nm. The detector is positionable to detect at least a portion of the light signals after transmission through the tissue under test (i.e. the transmitted light signals) and is operable to output a multiplexed signal indicative of an intensity of the transmitted light signals. In this regard, the drive signals may be configured to provide for multiplexing (e.g., time-division, wavelength-division, or code-division multiplexing) of the light signals. The drive signal generator is operable to supply the drive signals to the laser diodes, and the demodulator is operable to demodulate the multiplexed signal output by the detector to obtain signal portions corresponding with each of the transmitted light signals that are employable to determine a blood analyte level in the tissue under test.

The modulation frequency of each drive signal is set to cause its corresponding laser diode to operate in a low noise regime wherein intensity noise is reduced, and the modulation depth of each drive signal is set to broaden the line width of the laser diode and thereby reduce the potential for noise from optical feedback. In this regard, the modulation frequency and depth of each drive signal are set to achieve operation of its corresponding laser diode at a desired laser intensity noise level. In this regard, the desired laser intensity noise level may be near that (e.g., within the same order of magnitude) of the independent (i.e., outside of the system) laser relative intensity noise (RIN) level, typically approximately −120 dB/Hz over a predetermined measurement bandwidth.

More particularly, the modulation frequency of each drive signal may be between a lower frequency limit corresponding to a −3 db point on a 1/f noise versus frequency curve of the apparatus and an upper frequency limit corresponding to a relaxation oscillation frequency of its corresponding laser diode. In this regard, the modulation frequency may be in the range of 500 Hz to 10 Ghz, and, more preferably, is in the range of 1 kHz to 100 MHz.

The modulation depth of each drive signal may be such that their corresponding laser diodes are modulated until just above their threshold levels for lasing operation in order to achieve the largest possible line width broadening while still causing lasing operation, and, thus, achieve the greatest reduction in susceptibility to optical feedback. In this regard the minimum current level of each drive signal at least exceeds a threshold current for lasing operation of its corresponding laser diode. If the resulting laser line width of one of the laser diodes exceeds the line width specification of the apparatus, the modulation depth of its corresponding drive signal may be lessened until the laser diode has a broadened line width within the line width specification of the apparatus. As an alternative, shallow modulation may be employed to achieve better system accuracy at the expense of an increased susceptibility to optical feedback. Regarding shallow modulation, the modulation depth of each drive signal may be in the range of 0.1 percent to 10 percent of the total signal level of the drive signal.

According to another aspect of the present invention, a method for use in photoplethysmographic measurement of a blood analyte level in a tissue under test includes the step of transmitting a plurality of light signals at different predetermined center wavelengths through the tissue under test. Transmission of the light signals is accomplished by driving a corresponding plurality of laser diodes with a corresponding plurality of drive signals. At least a portion of the light signals (i.e. the transmitted light signals) are detected. A multiplexed signal indicative of an intensity of the transmitted light signals is output. The multiplexed signal is demodulated to output signal portions corresponding with each of the light signals that are employable to determine a blood analyte level in the tissue under test.

In the step of transmitting, each drive signal used to drive the laser diodes has a particular modulation frequency and modulation depth. The modulation frequency and depth of each drive signal is set to achieve operation of its corresponding laser diode at a desired laser intensity noise level. In this regard, the desired laser intensity noise level may be near that (e.g., within the same order of magnitude) of the independent laser RIN level, typically approximately −120 dB/Hz over a predetermined measurement bandwidth. More particularly, the modulation frequency of each drive signal may be between a lower frequency limit corresponding to a −3 db point on a 1/f noise versus frequency curve of the apparatus and an upper frequency limit corresponding to a relaxation oscillation frequency of its corresponding laser diode. In this regard, the modulation frequency may be in the range of 500 Hz to 10 GHz, and, more preferably is in the range of 1 kHz to 100 MHz.

The photoplethysmographic measurement apparatus and related method of the present invention achieve several advantages in addition to the reduction of noise. By modulating the laser diodes relatively fast, shortened effective laser on-times are achieved thereby reducing the possibility of laser diode damage due to thermal effects. If a slow response automatic power control (APC) drive circuit is used to compensate for laser output power changes due, for example, to changes in ambient temperature and aging of the laser diodes, the need for external temperature control (e.g., via a Peltier effect cooling device) of the laser diodes may be eliminated. The elimination of the need for external cooling of the laser diodes reduces the power requirements of the apparatus thereby increasing battery life in a battery powered apparatus. The removal of a Peltier effect cooler or the like also reduces the amount of heat that must be removed from a probe or other device in which the laser diodes may be included.

These and other aspects and advantages of the present invention will become apparent to one skilled in the art based upon further consideration of the following description.

### DESCRIPTION OF THE DRAWINGS

FIG. 1 shows a block diagram of one embodiment of a photoplethysmographic measurement apparatus in accordance with the present invention;

FIG. 2 shows a plot of one cycle of four exemplary drive signals that may be used for time-division multiplexing of light signals in the photoplethysmographic measurement apparatus of FIG. 1;

FIG. 3 shows a plot of a typical 1/f noise versus frequency curve for the photoplethysmographic measurement apparatus that is useful in identifying a lower modulation frequency limit for the drive signals of FIG. 2;

FIG. 4 shows a plot of one cycle of four additional exemplary drive signals that may be used for wavelength-division multiplexing of light signals in the photoplethysmographic measurement apparatus of FIG. 1; and

FIG. 5 shows a plot of one cycle of four more exemplary drive signals that may be used for code-division multiplexing of light signals in the photoplethysmographic measurement apparatus of FIG. 1.

### DETAILED DESCRIPTION

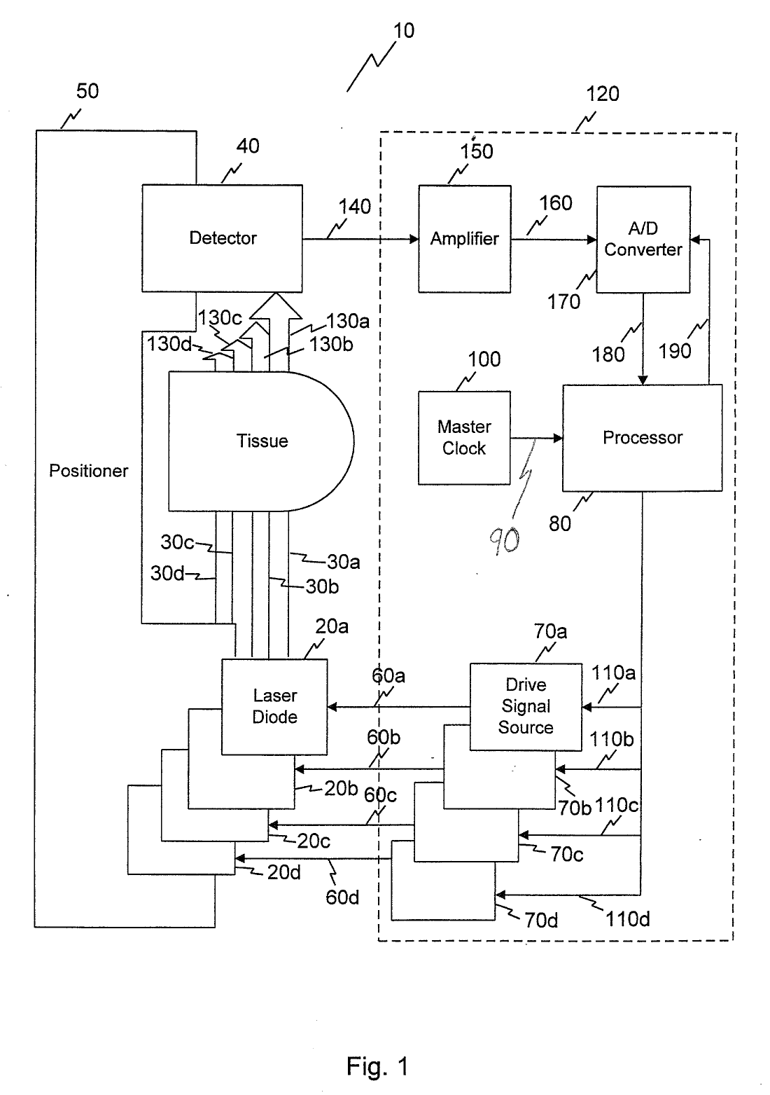

Referring now to FIG. 1, there is shown a block diagram of one embodiment of a photoplethysmographic measurement apparatus 10 in accordance with the present invention. The photoplethysmographic measurement apparatus 10 is configured for use in determining one or more blood analyte levels in a tissue under test, such as O2Hb, RHb, COHb and MetHb levels. The apparatus 10 includes a plurality of laser diodes 20a-d for emitting a corresponding plurality of light signals 30a-d centered at different predetermined center wavelengths λ₁, λ₂, λ₃, λ₄ through the tissue under test and on to a detector 40 (e.g., a photo-sensitive diode). The center wavelengths λ₁, λ₂, λ₃, λ₄ required depend upon the blood analytes to be determined. For example, in order to determine the levels of O2Hb, RHb, COHb and MetHb, λ₁ may be about 640 nm, λ₂ may be about 660 nm, λ₃ may be about 800 nm, and λ₄ may be about 940 nm. It should be appreciated that the present invention may be readily implemented with fewer or more laser diodes depending upon the number of different blood analyte levels to be measured.

The laser diodes 20a-d and detector 40 may be included in a positioning device 50, or probe, to facilitate alignment of the light signals 30a-d with the detector 40. For example, the positioning device 50 may be of clip-type or flexible strip configuration adapted for selective attachment to a patient's appendage (e.g., a finger).

The laser diodes 20a-d are activated by a corresponding plurality of analog drive signals 60a-d to emit the light signals 30a-d. The drive signals 60a-d are supplied to the laser diodes 20a-d by a corresponding plurality of drive signal sources 70a-d. The drive signal sources 70a-d may connected with a digital processor 80, which is driven with a clock signal 90 from a master clock 100. The digital processor 80 may be programmed to define modulation waveforms, or drive patterns, for each of the laser diodes 20a-d in accordance with predetermined values from a look-up table. More particularly, the digital processor 80 may provide separate digital trigger signals 110a-d to the drive signal sources 70a-d, which in turn generate the analog drive signals 60a-d.

The drive signal sources 70a-d, processor 80 and clock 100 may all be housed in a monitor unit 120. While the illustrated embodiment shows the laser diodes 20a-d physically interconnected with the positioning device 50 (e.g., mounted within the positioning device 50 or mounted within a connector end of a cable that is selectively connectable with the positioning device 50), it should be appreciated that the laser diodes 20a-d may also be disposed within the monitor unit 120. In the latter case, the light signals 30a-d emitted from the laser diodes 20a-d may be directed from the monitor unit 120 via one or more optical fibers to the positioning device 50 for transmission through the tissue. Furthermore, the drive signal sources 70a-d may comprise a single drive signal generator unit that supplies each of the drive signals 60a-d to the laser diodes 20a-d.

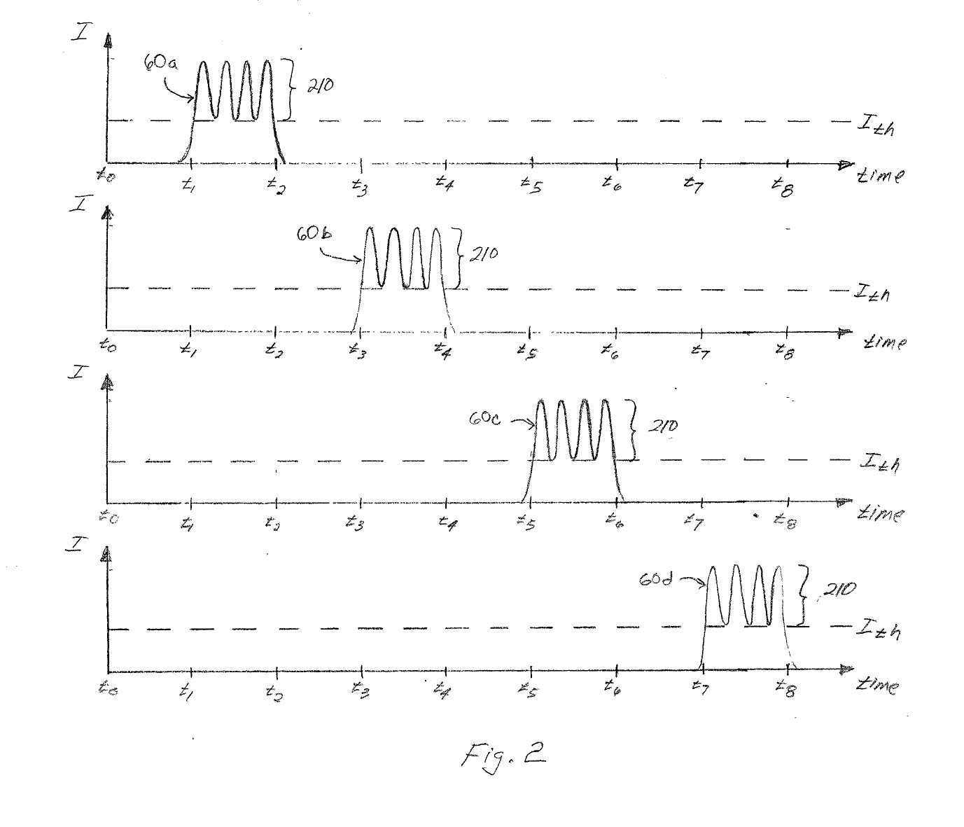

Transmitted light signals 130a-d (i.e., the portions of light signals 30a-d exiting the tissue) are detected by the detector 40. The detector 40 detects the intensities of the transmitted signals 130a-d and outputs a current signal 140 wherein the current level is indicative of the intensities of the transmitted signals 130a-d. As may be appreciated, the current signal 140 output by the detector 40 comprises a multiplexed signal in the sense that it is a composite signal including information about the intensity of each of the transmitted signals 130a-d. Depending upon the nature of the drive signals 60a-d, the current signal 140 may, for example, be time-division multiplexed, wavelength-division multiplexed, or code-division multiplexed, as will be further discussed below in connection with FIGS. 2, 4 and 5. It will be appreciated that the detector 40 must operate fast enough to detect the modulation frequencies to be demultiplexed.

The current signal 140 is directed to an amplifier 150, which may be housed in the monitor unit 120 as is shown. As an alternative, the amplifier 150 may instead be included in a probe/cable unit that is selectively connectable with the monitor unit 120. The amplifier 150 converts the current signal 140 to a voltage signal 160 wherein a voltage level is indicative of the intensities of the transmitted signals 130a-d. The amplifier 150 may also be configured to filter the current signal 140 from the detector 40 to reduce noise and aliasing. By way of example, the amplifier 150 may include a bandpass filter to attenuate signal components outside of a predetermined frequency range encompassing modulation frequencies of the drive signals 60a-d.

Since the current signal 140 output by the detector 40 is a multiplexed signal, the voltage signal 160 is also a multiplexed signal, and thus, the voltage signal 160 must be demultiplexed in order to obtain signal portions corresponding with the intensities of the transmitted light signals 130a-d. In this regard, the digital processor 80 may be provided with demodulation software for demultiplexing the voltage signal 160. In order for the digital processor 80 to demodulate the voltage signal 160, it must first be converted from analog to digital. Conversion of the analog voltage signal 160 is accomplished with an analog-to-digital (A/D) converter 170, which may also be included in the monitor unit 120. The A/D converter 170 receives the analog voltage signal 160 from the amplifier 150, samples the voltage signal 160, and converts the samples into a series of digital words 180 (e.g., eight, sixteen or thirty-two bit words), wherein each digital word is representative of the level of the voltage signal 160 (and hence the intensities of the transmitted light signals 130a-d ) at a particular sample instance. In this regard, the A/D converter 170 should provide for sampling of the voltage signal 160 at a rate sufficient to provide for accurate tracking of the shape of the various signal portions comprising the analog voltage signal 160 being converted. For example, the A/D converter 170 may provide for a sampling frequency at least twice the frequency of the highest frequency drive signal 60a-d, and typically at an even greater sampling rate in order to more accurately represent the analog voltage signal.

The series of digital words 180 is provided by the A/D converter 170 to the processor 80 to be demultiplexed. More particularly, the processor may periodically send an interrupt signal 190 (e.g., once per every eight, sixteen or thirty-two clock cycles) to the A/D converter 170 that causes the A/D converter 170 to transmit one digital word 180 to the processor 80. The demodulation software may then demultiplex the series of digital words 180 in accordance with an appropriate method (e.g., time, wavelength, or code) to obtain digital signal portions indicative of the intensities of each of the transmitted light signals 130a-d.

Referring now to FIG. 2 there is shown one cycle of four exemplary drive signals 60a-d that may be supplied by the drive signal sources 70a-d to cause the laser diodes 20a-d to emit light signals 30a-d. Each of the drive signals 60a-d comprises a non-zero sine wave for a limited period of time during each cycle. More specifically, during the periods of time when the drive signals 60a-d are non-zero (i.e., the on periods), the current level of each drive signal 60a-d exceeds a threshold current level Iₜₕ for lasing operation of its corresponding laser diode 20a-d (Iₜₕ may be different for each of the laser diodes 20a-d ). The on periods may be sequenced in time so that the light signals 30a-d emitted by the laser diodes 20a-d are time-division multiplexed. For example, during one cycle from time t₀ to t₈, drive signal 60a may be on between times t₁ and t₂, drive signal 60b may be on between times t₃ and t₄, drive signal 60c may be on between times t₅ and t₆, and drive signal 60d may be on between times t₇ and t₈. Between the on periods there are dark periods (t₀ to t₁, t₂ to t₃, t₄ to t₅, and t₆ to t₇). System noise can be measured during the dark periods and subtracted from the on period signals to remove system noise.

Noise introduced to the light signals 30a-d by operation of the laser diodes 20a-d is reduced by setting two parameters of the drive signals 60a-60d: modulation frequency and modulation depth. The modulation frequency of each drive signal 60a-d is chosen so that its corresponding laser diode 20a-d operates in a low noise regime wherein laser intensity noise as a result of heating of the laser diode 20a-d during operation is reduced. In this regard, the modulation frequency and depth of each drive signal 60a-d is set to cause its corresponding laser diode 20a-d to operate with a laser intensity noise level near that (e.g., within the same order of magnitude) of the independent laser RIN level, typically approximately −120 dB/Hz over a predetermined measurement bandwidth. For example, in instances where the laser intensity noise level increases 10% when used in the apparatus 10, the modulation frequency and depth of each drive signal 60a-d may be set to achieve operation of its corresponding laser diode 20a-d to operate at a laser intensity noise level within 5% of the independent laser RIN level.

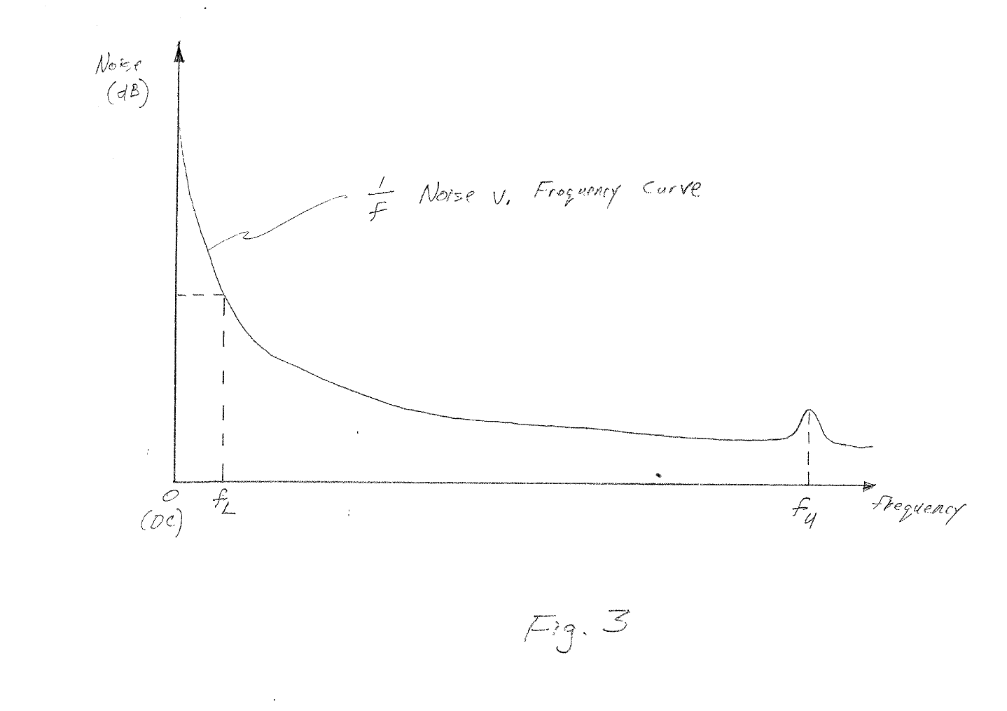

Referring now to FIG. 3, the modulation frequency of each drive signal 60a-d during their respective on periods required to achieve operation of the laser diodes 20a-d in the desired low noise regime will typically be greater than a lower frequency limit f_{L} corresponding with the −3 db point on the 1/f noise versus frequency curve of the photophethysmographic measurement apparatus. In this regard, the modulation frequency of each drive signal 60a-d is preferably at least 500 Hz, and more preferably, is at least 1 kHz. Likewise, the modulation frequency of each drive signal 60a-d required to achieve operation of the laser diodes 20a-d in the desired low noise regime will typically be less than an upper frequency limit f_{U} corresponding with the relaxation oscillation frequency of its corresponding laser diode 20a-d. The relaxation oscillation frequency (i.e. the frequency of oscillations in the intensity of the light signal output by a laser diode before reaching stable operation after being activated) of the laser diodes 20a-d will be dependent upon the structural properties of the laser diodes 20a-d and must be empirically determined, but will typically exceed 1 GHz. In this regard, the modulation frequency of each drive signal 60a-d is preferably no higher than 10 GHz, and more preferably, is no higher than 100 MHz. Federal Communications Commission (FCC) and electromagnetic interference (EMI) limitations may also be considered when choosing the appropriate modulation frequency for each drive signal 60a-d. It should be appreciated that the modulation frequencies of each drive signal 60a-d may be different. Furthermore, the detector 40 must be sufficiently fast to detect each drive signal 60a-d at the modulation frequencies chosen.

Referring again to FIG. 2, the modulation depth 210 (i.e. the peak-to-peak power) of each drive signal 60a-d determines how much the line width of its corresponding laser diode 20a-d is broadened. Broadening the line width of the laser diodes 20a-d reduces their coherence, and thus reduces their susceptibility to optical feedback noise. In general, the modulation depth 210 of each drive signal 60a-d may be set so that its corresponding laser diode 20a-d operates with a broadened line width wherein the noise level of each laser diode 20a-d approaches its independent laser RIN level. In this regard, the modulation depth 210 of each drive signal 60a-d may be set to modulate its corresponding laser diode 20a-d until just above the threshold current Iₜₕ as is shown in FIG. 2. This achieves line width broadening during lasing operation of the laser diodes 20a-d, and thus a reduction in susceptibility to optical feedback noise.

The photoplethysmographic measurement apparatus 10 may have a predetermined line width specification which is narrower than the line width achieved with the largest modulation depth 210. For example, in order to accurately determine blood analyte levels from the transmitted light signals 130a-d, it may be specified that the line width of the laser diodes 20a-d be no greater than 3 nm (measured at full-width, half-maximum power). The modulation depth 210 of the drive signals 60a-d may be accordingly lessened to achieve broadened line widths within the predetermined line width specification. In this regard, the deepest modulation depth 210 possible while still remaining within the predetermined line width specification is used to achieve the greatest possible reduction in noise from optical feedback. Further, it should be appreciated that the modulation depth 210 of each drive signal 60a-d may set to the same or different amounts.

Alternatively, a modulation depth 210 shallower than possible within the predetermined line width specification may instead be used to increase overall system accuracy at the expense of less reduction in noise from optical feedback. While any modulation depth 210 that is detectable by the detector 40 may be used, typical appropriate shallow modulation depths range from 0.1 to 10 percent of the total signal level of the corresponding drive signal 60a-d.

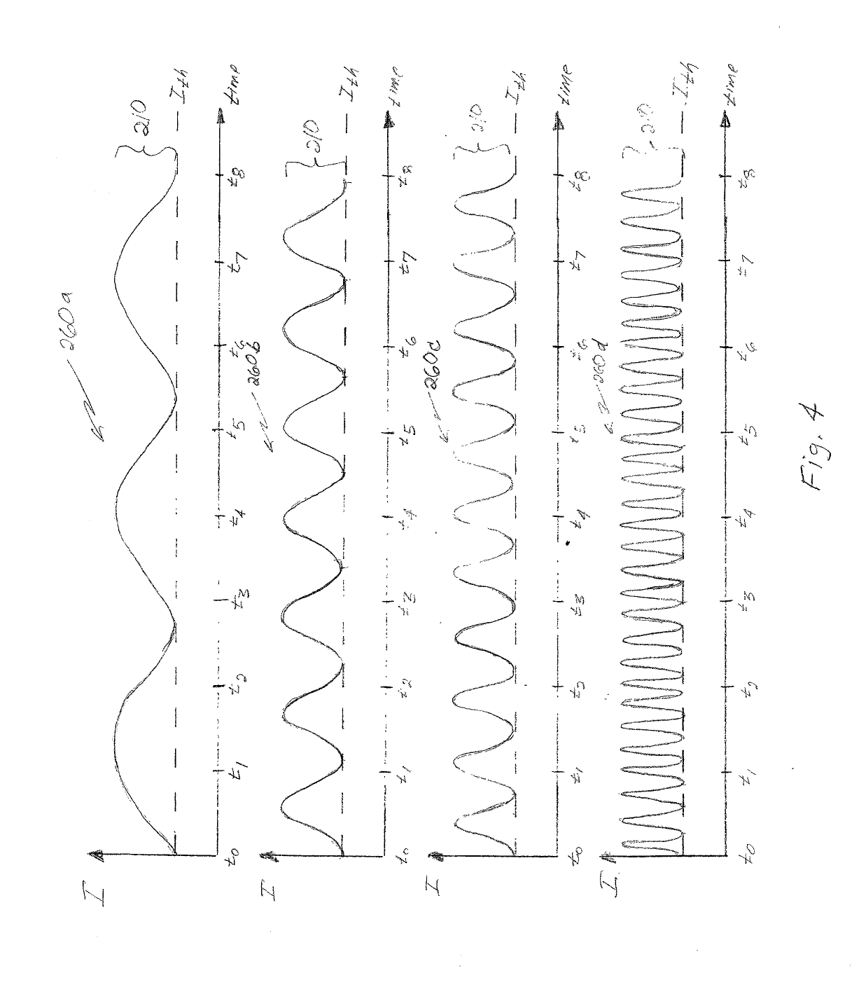

In addition to time division multiplexing of the drive signals 60a-d, other multiplexing techniques may be employed so that the intensity of each of the transmitted signals 130a-d may be obtained from the current signal 140 output by the detector 40. For example, FIG. 4 shows four different exemplary drive signals 260a-d appropriate for wavelength-division multiplexing of the light signals 30a-d. Each drive signal 260a-d is modulated at a modulation frequency that is orthogonal to the modulation frequency of each of the other drive signals 260a-d. For example, the four drive signals 260a-d may be modulated at modulation frequencies wherein there are 3, 7, 11, and 29 cycles of each drive signal 260a-d during the time period from t₀ to t₈. In this manner, wavelength-division multiplexing of the light signals 30a-d is achieved, and the current signal 140 from the detector 40 may be demultiplexed accordingly to obtain the intensities of each transmitted light signal 130a-d.

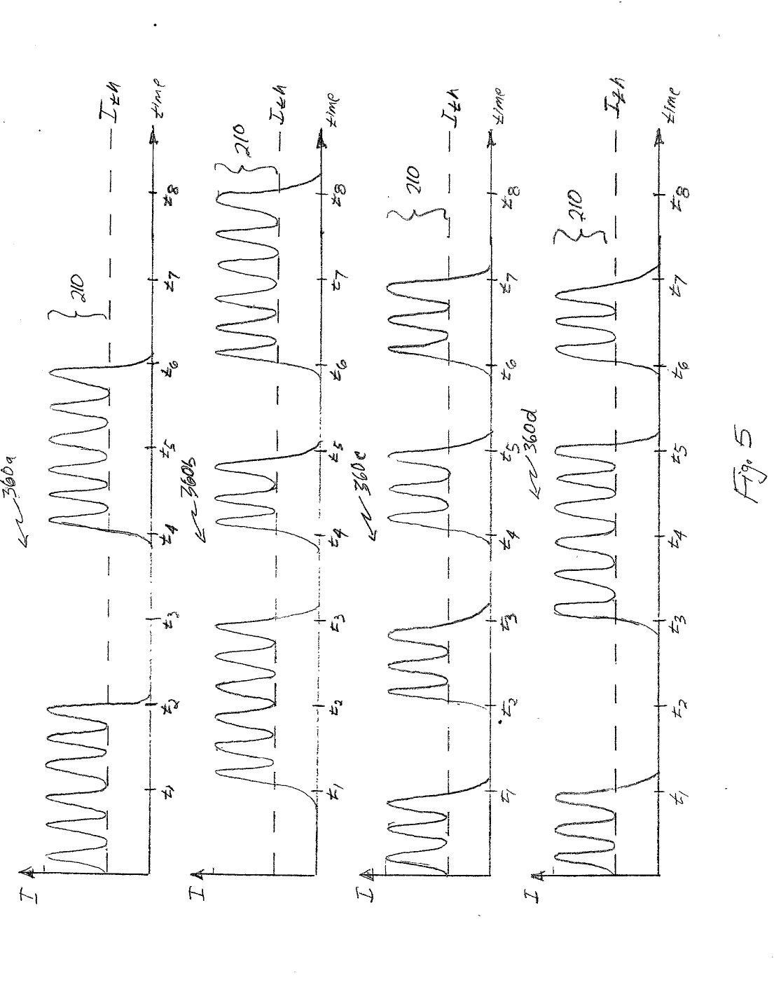

As another example, FIG. 5 shows one cycle of four exemplary drive signals 360a-d that are appropriate for code-division multiplexing of the light signals 30a-d. Each of the drive signals 360a-d comprises a non-zero sine wave exceeding the threshold current level Iₜₕ for lasing operation of its corresponding laser diode 20a-d that has been multiplied by a square wave signal representing a unique binary code associated with its corresponding laser diode 20a-d. For example, drive signal 360a may be obtained by multiplying a sine wave with a square wave representing the 8-bit binary sequence 11001100, drive signal 360b may be obtained by multiplying a sine wave with a square wave representing the 8-bit binary sequence 01101011, drive signal 360c may be obtained by multiplying a sine wave with a square wave representing the 8-bit binary sequence 10101010, and drive signal 360d may be obtained by multiplying a sine wave with a square wave representing the 8-bit binary sequence 10011010. By multiplying the multiplexed current signal 140 output by the detector 40, by the appropriate binary sequence, the intensity of each transmitted light signal 130a-d may be obtained.

While an embodiment of the present invention having four laser diodes and four drive signals has been described in detail, further modifications and adaptations of the invention may occur to those skilled in the art. However, it is expressly understood that such modifications and adaptations are within the spirit and scope of the present invention.

What is claimed is:

1. A photoplethysmographic measurement apparatus for determining a blood analyte level in a tissue under test, said apparatus comprising:
a plurality of laser diodes operable to transmit a corresponding plurality of light signals centered at different predetermined wavelengths through the tissue under test in response to a corresponding plurality of drive signals;
a detector positionable to detect at least a portion of said light signals after transmission through the tissue under test and operable to output a multiplexed signal indicative of an intensity of said detected portion of said light signals;
a drive signal generator operable to supply said drive signals to said laser diodes, wherein each said drive signal includes a modulation frequency and a modulation depth, wherein said modulation frequency and modulation depth of each said drive signal are set to achieve operation of its corresponding laser diode at a desired laser intensity noise level; and
a demodulator operable to demodulate said multiplexed signal to obtain signal portions corresponding with each of said light signals, wherein said signal portions are employable to determine a blood analyte level in the tissue under test.

2. The apparatus of claim 1 wherein said desired laser intensity noise level is within the same order of magnitude as that of an independent laser RIN level of said corresponding laser diode.

3. The apparatus of claim 1 wherein said modulation frequency of each said drive signal is between a lower frequency limit corresponding to a −3 db point on a 1/f noise versus frequency curve of said photoplethysmographic measurement apparatus and an upper frequency limit corresponding to a relaxation oscillation frequency of its corresponding laser diode.

4. The apparatus of claim 3 wherein said modulation frequency of each said drive signal is in the range of 500 Hz to 10 Ghz.

5. The apparatus of claim 3 wherein said modulation frequency of each said drive signal is in the range of 1 kHz to 100 MHz.

6. The apparatus of claim 1 wherein there are first and second laser diodes, and wherein said first laser diode is operable to transmit a first light signal centered at a first predetermined wavelength in the range of 600 nm to 700 nm and said second laser diode is operable to transmit a second light signal centered at a second predetermined wavelength in the range of 900 nm to 1000 nm.

7. The apparatus of claim 1 wherein said modulation depth of each said drive signal provides a drive signal having a minimum current level at least exceeding a threshold current for lasing operation of its corresponding laser diode.

8. The apparatus of claim 7 wherein said modulation depth of each said drive is in the range of 0.1 percent to 10 percent of a total signal level of each said drive signal.

9. The apparatus of claim 1 wherein each said drive signal comprises a sine wave having a modulation frequency orthogonal to said modulation frequencies of said other drive signals, whereby said multiplexed signal comprises a wavelength division multiplexed signal.

10. The apparatus of claim 1 wherein each said drive signal comprises a sine wave having a minimum amplitude exceeding a threshold current of its corresponding laser diode for only a predetermined temporal period, and wherein said predetermined temporal periods of each said drive signal are sequenced in time, whereby said multiplexed signal comprises a time division multiplexed signal.

11. The apparatus of claim 1 wherein each said drive signal comprises a sine wave having a minimum amplitude exceeding a threshold current of its corresponding laser diode multiplied with a square wave signal, and wherein each said square wave signal represents a unique binary code associated with its corresponding laser diode, whereby said multiplexed signal comprises a code division multiplexed signal.

12. A photoplethysmographic measurement apparatus for determining a blood analyte level in a tissue under test, said apparatus comprising:
a plurality of laser diodes for transmitting a corresponding plurality of light signals centered at different predetermined wavelengths through the tissue under test, wherein each said laser diode is modulated by a corresponding drive signal having a modulation frequency between a lower frequency limit corresponding to a −3 db point on a 1/f noise versus frequency curve of said photoplethysmographic measurement apparatus and an upper frequency limit corresponding to a relaxation oscillation frequency of its corresponding laser diode;
a detector for detecting at least a portion of said light signals after transmission through the tissue under test and outputting a multiplexed signal indicative of an intensity of said detected portion of said light signals; and
a demodulator for demodulating said multiplexed signal to output signal portions corresponding with each of said light signals, wherein said signal portions are employable to determine a blood analyte level in the tissue under test.

13. The apparatus of claim 12 wherein said modulation frequency of each said drive signal is in the range of 500 Hz to 10 Ghz.

14. The apparatus of claim 12 wherein said modulation frequency of each said drive signal is in the range of 1 kHz to 100 MHz.

15. The apparatus of claim 12 wherein each said drive signal has a modulation depth, and wherein said modulation depth of each said drive signal provides a drive signal having a minimum current level at least exceeding a threshold current for lasing operation of its corresponding laser diode.

16. The apparatus of claim 15 wherein said modulation depth of each said drive is in the range of 0.1 percent to 10 percent of a total signal level of each said drive signal.

17. A method for use in photoplethysmographic measurement of a blood analyte level in a tissue under test, said method comprising:
transmitting a plurality of light signals at different predetermined center wavelengths through the tissue under test by driving a corresponding plurality of laser diodes with a corresponding plurality of drive signals, wherein each drive signal has a modulation frequency and a modulation depth, and wherein the modulation frequency and modulation depth of each drive signal are set to achieve operation of its corresponding laser diode at a desired laser intensity noise level;
detecting at least a portion of the light signals;
outputting a multiplexed signal indicative of an intensity of the detected portion of the light signals; and
demodulating the multiplexed signal to output signal portions corresponding with each of the light signals, wherein the signal portions are employable to determine a blood analyte level in the tissue under test.

18. The method of claim 17 wherein the desired laser intensity noise level is within the same order of magnitude as that of an independent laser RIN level of the corresponding laser diode.

19. The method of claim 17 wherein in said step of transmitting, the modulation frequency of each drive signal is between a lower frequency limit corresponding to a −3 db point on a 1/f noise versus frequency curve of a system used to transmit the light signals and an upper frequency limit corresponding to a relaxation oscillation frequency of its corresponding laser diode.

20. The method of claim 19 wherein in said step of transmitting, the modulation frequency of each drive signal is in the range of 500 Hz to 10 Ghz.

21. The method of claim 19 wherein in said step of transmitting, the modulation frequency of each drive signal is in the range of 1 kHz to 100 MHz.

22. The method of claim 17 wherein in said step of transmitting, a first light signal centered at a first predetermined wavelength in the range of 600 nm to 700 nm and a second light signal centered at a second predetermined wavelength in the range of 900 nm to 1000 nm are transmitted.

23. The method of claim 17 wherein in said step of transmitting, the modulation depth of each drive signal provides a drive signal having a minimum current level at least exceeding a threshold current for lasing operation of its corresponding laser diode.

24. The method of claim 23 wherein in said step of transmitting, the modulation depth of each drive signal is in the range of 0.1 percent to 10 percent of a total signal level of each drive signal.

25. The method of claim 17 wherein in said step of transmitting, each drive signal comprises a sine wave having a modulation frequency orthogonal to the modulation frequencies of the other drive signals, whereby, in said step of outputting the multiplexed signal comprises a wavelength division multiplexed signal.

26. The method of claim 17 wherein in said step of transmitting, each drive signal comprises a sine wave having a minimum amplitude exceeding a threshold current of its corresponding laser diode for only a predetermined temporal period, and wherein the predetermined temporal periods of each of the drive signals are sequenced in time, whereby in said step of outputting, the multiplexed signal comprises a time division multiplexed signal.

27. The method of claim 17 wherein in said step of transmitting, each drive signal comprises a square wave signal, and wherein each square wave signal represents a unique binary code associated with its corresponding laser diode, whereby in said step of outputting, the multiplexed signal comprises a code division multiplexed signal.

28. An apparatus for driving a plurality of laser diodes in a photoplethysmographic probe, said apparatus comprising:
a drive signal generator operable to supply each of the laser diodes with a corresponding drive signal, wherein each said drive signal has a modulation frequency and a modulation depth, wherein said modulation frequency and modulation depth of each said drive signal are set to achieve operation of its corresponding laser diode at a desired laser intensity noise level.

29. The apparatus of claim 28 wherein said desired laser intensity noise level is within the same order of magnitude as that of an independent laser RIN level of said corresponding laser diode.

30. The apparatus of claim 28 wherein said modulation frequency of each said drive signal is between a lower frequency limit corresponding to a −3 db point on a 1/f noise versus frequency curve of said photoplethysmographic probe and an upper frequency limit corresponding to a relaxation oscillation frequency of its corresponding laser diode.

31. The apparatus of claim 28 wherein said modulation frequency of each said drive signal is in the range of 500 Hz to 10 Ghz.

32. The apparatus of claim 28 wherein said modulation frequency of each said drive signal is in the range of 1 kHz 100 MHz.
